(19) United States
(12) Patent Application Publication
(10) Pub. No.: US 2002/0128499 A1
(43) Pub. Date: Sep. 12, 2002

(54) 2-Thio-5-amino substituted benzoquinones, their manufacture and their use in rubber

(76) Inventors: Otto William Maender, Copley, OH (US); Charles John Rostek, Chagrin Falls, OH (US); Alan Roy Katritzky, Gainesville, FL (US); Herman Howard Odens, Wilmington, DE (US); Michael Victor Voronkov, Cranbury, NJ (US)

Correspondence Address: Louis A. Morris, Akzo Nobel Inc., 7 Livingstone Avenue, Dobbs Ferry, NY, 10522-3408, US

(21) Appl. No.: 10/004,659

(22) Filed: Dec. 5, 2001

Related U.S. Application Data
(60) Provisional application No. 60/251,745, filed on Dec. 5, 2000.

(51) Int. Cl. C07C 251/14

(52) U.S. Cl. 552/297; 552/301

(57) ABSTRACT

The invention comprises a process for preparing a compound having the formula comprising:

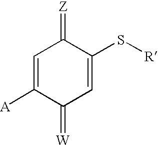

where Z and W are the same or different, and selected from the group consisting of O, NH and NR, where R is H, alkyl, cycloalkyl or aryl, R′ is alkyl, cycloalkyl, alkylene, alkyl 3-propionate, aryl or arylene. A is H if either or both of Z and W are not O, but if both Z and W are O, A is:

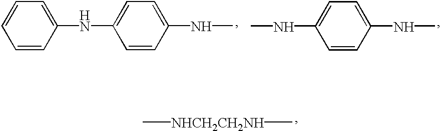

or a carbon based heterocyclic component having an amine constituent, or RR″N-, where R″ is the same or different than R and is selected from the same group as R. The process comprises preparing an alkylthio-substituted benzoquinone intermediate by reacting in a first reaction mixture a thio-substituted aromatic having the formula:

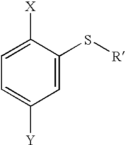

where X is OH, NH₂ or NHR when Z is O, NH or NR, respectively, and where Y is OH, NH₂ or NHR when W is O, NH or NR, respectively, with an alkali metal periodate in the presence of a phase transfer catalyst, followed, if A is not H, by reacting the intermediate in a second reaction mixture containing an amine included in the group of amines comprising A, and in the presence of oxygen to obtain the compound. If A is H no second reaction mixture is employed and the intermediate comprises the compound. The invention includes a process for making the composition and the use of the composition in natural and synthetic rubber, and with monomers, gasoline and lubricating oil to inhibit polymerization.

### CROSS REFERENCE TO RELATED APPLICATION

This application claims priority from Provisional Patent Application 60/251,745, filed Dec. 5, 2000, the content of which is incorporated herein in its entirety.

### BACKGROUND OF THE INVENTION

1. Field of the Invention

The invention relates to 2-thio-5-amino substituted benzoquinones, their manufacture and their use in rubber as antidegradants.

2. Discussion of the Prior Art

Vulcanizing rubber compositions by heating a sulfur-vulcanizable rubber composition with sulfur and/or a sulfur donor and a vulcanization accelerator has been known for many years. By this process, vulcanizates having acceptable physical properties including tensile strength, resilience, and fatigue resistance can be obtained, but such vulcanizates tend not to have good aging properties.

Uncured as well as cured rubbers are prone to aging effects. The unsaturated groups in diene rubbers, e.g. styrene-butadiene rubber (SBR) or a blend of SBR with natural rubber, butadiene rubber or with both, make it possible to cure with sulfur, but at the same time they exhibit a sensitivity toward oxygen, ozone, and other reactive substances causing changes such as hardening of the vulcanizate. Unaged diene rubbers contain free double bonds that remain sensitive to the above reactive substances even after vulcanization. Higher temperatures make these effects even more noticeable.

Protective agents are used to protect the rubber vulcanizate against various forms of aging, fatigue, and ozone. For example, exposure of pneumatic tires to ozone leads to the formation of ozone cracks, in particular in the side walls of the tire. A well-known class of protective agents are N,N′-disubstituted, in particular N-alkyl-N′-phenyl p-phenylenediamine derivatives. These N,N′-disubstituted p-phenylenediamine derivatives typically are also referred to as antidegradants, antiozonants or antioxidants. The reader is directed to Hofmann, Rubber Technology Handbook, Hanser Publishers, Munich 1989, pp. 264-277, in particular pp. 269-270. These antidegradants are commercially available inter alia under the trademark Santoflex® sold by Flexsys America LP. In the rubber industry, the most frequently used antidegradant is N-(1,3-dimethylbutyl)-N′-phenyl-p-phenylenediamine or 6PPD.

There are known strategies for the preparation of 2-thio-5-amino substituted benzoquinones. In the first and previously more generally applied approach, the nitrogen substituent is introduced first usually via displacement of a halogen or an alkoxy group from the appropriate quinone. The thiol moiety is then introduced by displacement of another halide or alkoxy moiety on the same quinone (see Petersen, S.; Gauss, W.; Urbschat, E., Angew. Chem., 1955, 67, 217.; Gauss, W.; Petersen, S. Angew. Chem., 1957, 69, 252; Marxer, A. Helv. Chim. Acta, 1957, 502) or by an addition/oxidation route (see Cajipe, G.; Rutolo, D.; Moor, H. W. Tetrahedron Lett., 1973, 474695).

In a second somewhat less widely accepted strategy, the nitrogen substituent is introduced into an alkylthio-substituted benzoquinone via amine displacement of a leaving group, usually an alkoxide. Excess of the amine can introduce a second amino substituent (Vorkapic-Furac, J.; Kishi, H.; Porter, T. H.,

Folkers, K., Acta Pharm. Suec., 1977, 14, 171).

Both of the above strategies require the presence of at least two good leaving groups in the starting quinone, thus limiting their scope and accessibility. Surprisingly, no reference was found for the preparation of 2-thio-5-amino substituted benzoquinones by sequential introduction of both substituents via an addition/oxidation protocol.

Heavy metal oxides such a lead dioxide¹ and silver oxide² have been used in stoichiometric quantities to generate simple quinone diimines, e.g. N,N′-diphenyl-p-quinone diimine and N-(1,3-dimethylbutyl)-N-phenyl-p-quinone diimine in acetone or toluene solvents. (¹Campbell and Harmon, Monsanto Internal Reports (1969); ²Honzl and Metalová, Tetrahedron, Vol. 25, 3648 (1969).) Similarly, N,N′-diphenyl-p-quinone diimine was prepared in a benzene solution contacted with aqueous potassium ferricyanide³ and later, a similar process was used to generate N,N′-substituted alkylaryl-p-quinone diimines⁴.

Other workers suggested using the more expensive silver oxide instead of potassium ferricyanide to accomplish conversion of substituted p-phenylenediamines to their respective p-quinone diimines⁴. (³Feichtmayr and Wuerstlin, Berichte der Bunsengesellschaft Bd. 67, p. 435 (1963); ⁴Kotulak et al., Collect, Czech. Chem. Commun. 48 (12), pp. 3384-3395 (1983).)

Red lead (lead tetraacetate) has been used in the oxidation of the benzenesulfonyl, p-toluenesulfonyl and methanesulfonyl derivatives of p-aminophenol to obtain the corresponding p-quinone imides (Adams and Looker, Quinone Imides. IV. P-Quinone Monosulfonimides, JALS 73, 1145 (1951)).

### SUMMARY OF THE INVENTION

In one embodiment, the present invention comprises a process for preparing a compound having the formula comprising:

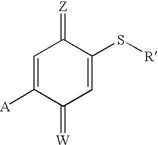

where Z and W are the same or different, and selected from the group consisting of O, NH and NR, where R is H, alkyl, cycloalkyl or aryl, R′ is alkyl, cycloalkyl, alkylene, alkyl 3-propionate, aryl or arylene. A is H if either or both of Z and W are not O, but if both Z and W are O, A is:

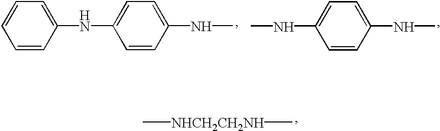

or a carbon based heterocyclic component having an amine constituent, or RR″N—, where R″ is the same or different than R and is selected from the same group as R. The process comprises preparing an alkylthio-substituted benzoquinone intermediate by reacting in a first reaction mixture a thio-substituted aromatic having the formula:

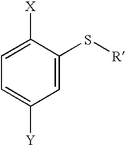

where X is OH, NH₂ or NHR when Z is O, NH or NR, respectively, and where Y is OH, NH₂ or NHR when W is O, NH or NR, respectively, with an alkali metal periodate in the presence of a phase transfer catalyst. If A is not H there is a following reaction of the intermediate in a second reaction mixture with an amine included in the group of amines comprising A, and in the presence of oxygen to obtain the compound. If A is H no second reaction mixture is employed and the intermediate comprises the compound.

In a second embodiment the present invention comprises a composition comprising the above compound.

In a third embodiment, the present invention comprises a composition comprising natural or synthetic rubber or a blend thereof and an antidegradant selected from the group consisting of thio-substituted amino-1,4-benzoquinones, 1,4-benzoquinoneimines, and 1,4-benzoquinonediimines.

In a fourth embodiment, the present invention comprises a composition comprising natural or synthetic rubber or a blend thereof and an antidegradant selected from 2-(4-anilinoanilino)-5-cyclohexylsulfanyl)-benzo-1,4-quinone, or 3-{[4-(4-anilinoanilino)-3,6-dioxo-1,4-cyclohexadien-1-yl]sulfanyl}propanoate, or a mixture thereof.

Other embodiments of the invention encompass specific compounds prepared by the above process, details concerning the process and process conditions and relative amounts of reactants and natural or synthetic rubber compositions, all of which are hereinafter disclosed in the following discussion of each of the facets of the present invention.

### DETAILED DESCRIPTION OF THE INVENTION

In the manufacture of alkylthio-substituted 1,4-aminobenzoquinones, 1,4-benzoquinoneimines and 1,4-benzoquinonediimines to be employed in the process of the present invention the order of addition of the thiol moiety and nitrogen substituent is important, because introduction frequently, although not invariably, results in bis adducts.

We have found alkali metal periodates to be surprisingly effective as oxidants for the above manufacture. The alkali metal periodate in the first reaction mixture may be used in either catalytic or stoichiometric amounts with respect to the thio-substituted aromatic. If in catalytic amounts an additional oxidant is added to the first reaction mixture in an amount sufficient to complete the oxidation of the thio substituted aromatic. The preferred additional oxidant is hydrogen peroxide.

The most preferred alkali metal periodate is sodium periodate.

The first reaction mixture of the process of the present invention may comprise organic and aqueous phases, since the oxidant will be introduced in aqueous solution. In that case a solvent will be employed in which the organic phase of the first reaction mixture is soluble. Following the reaction of the first reaction mixture, the organic phase dissolved in said solvent is separated from the aqueous phase and will comprise the second reaction mixture that will also include the amine and oxygen.

Preferred solvents are selected from the group consisting of methylene chloride, chlorobenzene, and C₁-C₄ alcohols (e.g. methanol, ethanol, and i-propyl alcohol).

The reaction conditions used with the first and second reaction mixtures comprise a temperature of from about 20° C. to about 25° C. The residence time for the first reaction mixture is from about 5 minutes to about 2 hours and for the second reaction mixture the residence time for is from about 2 hours to about 4 hours. The oxygen for the second reaction mixture is most easily provided by a constant stream of air.

It is preferred that the amine added to the second reaction mixture be 80% or less of the 1:1 stoichiometric amount required to produce the amino-1,4-benzoquinones. This will result in an amount of unreacted thio-substituted benzoquinone intermediate being added to the rubber composition with which the compound of the invention is used. Such intermediates have also been found to have anti-ozonant and anti-oxidant capabilities.

Preferred amines for use in the second reaction mixture are selected from the group consisting of aniline, 3-anisidine, 4-isopropylaniline, 2-toluidine, 2,4-dimethylaniline, 2-naphthylamine, 4-anilinoaniline, phenethylamine, N-methylaniline, morpholine, and indoline.

With water immiscible solvents, it is advantageous to utilize a phase transfer catalyst to accelerate the rate of reaction in the process of the present invention.

Phase transfer catalysts useable in the present invention include, but are not limited to, quaternary ammonium bases and salts, such as tetramethylammonium hydroxide, tetraalkylammonium halides such as benzyltrimethylammonium bromide, tetra-N-butyl ammonium bromide, tetra-N-butylammonium chloride, benzyltriethyl ammonium chloride; phosphonium salts such as bis[tris(dimethylamino)phosphine]iminium chloride; crown ethers and polyethylene glycols.

A particularly preferred first reaction mixture includes methylene chloride solvent, the phase transfer catalyst comprising benzyltrimethylammonium bromide and the alkali metal periodate is sodium periodate which is used in either stoichiometric or catalytic amounts with respect to the thio-substituted aromatic. If sodium periodate is used in catalytic amounts, hydrogen peroxide is the most preferred additional oxidant in an amount sufficient to complete the oxidation of the thio-substituted aromatic.

Most preferred compounds of the present invention are selected from the group consisting of thio-substituted amino 1,4-benzoquinones, thio-substituted 1,4-benzoquinoneimines, and thio-substituted 1,4-benzoquinonediimines.

In a preferred embodiment of the process of the present invention the compound made is a thio-substituted amino-1,4-benzoquinone having the formula comprising:

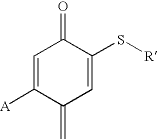

where A is:

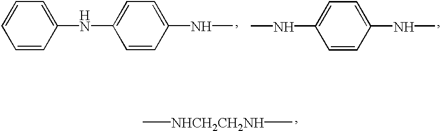

or a carbon based heterocyclic component having an amine constituent, or RR″N—, R′ is alkyl, cycloalkyl, alkylene, alkyl 3-propionate, aryl or arylene, R″ is the same or different than R and is selected from the same group as R. The thio-substituted amino-1,4-benzoquinone is obtained by reacting a thio-substituted 1,4-benzoquinone intermediate with a primary or secondary amine to obtain the thio-substituted amino-1,4-benzoquinone in accordance with the equation:

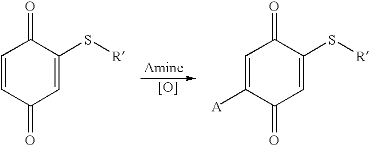

More preferred are thio-substituted amino-1,4-benzoquinones having the formula:

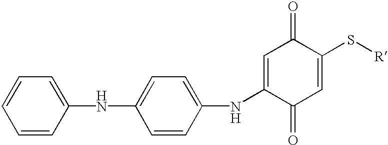

where, R′ is alkyl, cycloalkyl, alkylene, alkyl 3-propionate, aryl or arylene.

According to the present invention, it has been found that by adding appropriate amounts of the above products of the present invention to a vulcanizable rubber composition comprising natural rubber or other rubbers, vulcanizates, from which, e.g., pneumatic tires can be made, having anti-aging, fatigue, and ozone resistance properties can be obtained.

Either natural rubber (NR), styrene-butadiene rubber (SBR) or a blend of NR and SBR or NR and SBR with one or more other rubbers can be used in the invention process, it being understood that for purposes of this invention the term “rubber” means an elastomer containing a hydrocarbon unit which is a polymer with some unsaturated chemical bonds. Typically, SBR, a blend of SBR with natural rubber (NR), a blend of SBR with polybutadiene rubber or butadiene rubber (BR), or a blend of SBR with NR and BR is used. The type of rubber or mixture of rubbers will have some effect on the precise amounts of antidegradant to be used.

Typically, the amount of antidegradant employed in the rubber composition of the present invention will be at least about 0.5 phr. The preferred upper limit is about 5.0 phr, most preferably 3.0 phr.

In this application, the abbreviation “phr” means the number of parts by weight per 100 parts by weight of rubber. In the case of a rubber blend, it is based on 100 parts by weight of total rubber.

The natural or synthetic rubber or blend thereof may comprise a mixture of two or more antidegradants selected from the antidegradants of the present invention or one or more antidegradants selected from such antidegradants in combination with a non-thio antidegradant. The preferred non-thio-substituted antidegradants are selected from the group consisting of phenylenediamines, dihydroquinolines, and phenolics or a blend thereof.

In the case where the rubber is polybutadiene and the antidegradant is selected from the group consisting of alkylthio-amino-p-benzoquinones, alkylthio-p-quinoneimines, and alkylthio-p-quinonediimines, the oxidation induction time (OIT) of the antidegradant as measured by differential scanning calorimetry (DSC) at 140° C. should be at least about 5 minutes. Similarly, in polyisoprene, the oxidation induction time of the antidegradant at 160° C. should be at least about 8 minutes. The OIT procedure used to measure the efficiency of the respective antioxidants in polyisoprene and polybutadiene follows.

A sample of 0.5 wt. % antioxidant in polymer¹ is used for DSC oxidation induction time analysis. The sample is run on a TA Instruments 2910 differential scanning calorimeter equipped with nitrogen delivery at 30 ml/min and 100% oxygen delivery at 70 ml/min. An isothermal program is used at 140° C. in polybutadiene or at 160° C. in polyisoprene under oxygen until an oxidation exotherm is detected². The sample is first equilibrated at the respective temperatures under nitrogen. Oxygen is then turned on when the isothermal step of the program starts. The oxidation induction time is measured from the point when oxygen is turned on to the onset of the oxidation exotherm. ¹ cis Polyisoprene made from synthetic rubber Average Mw ca. 40,000 (GPC) from Aldrich 43-126-5, Polybutadiene average Mₙ 3,000 (VPO) 43,478-7 or combinations of both polymers were used. ² The isothermal temperature can be adjusted for an antioxidant/polymer system to give an exotherm which occurs between about 15 min. to an hour.

It is preferred that the alkyl, cycloalkyl, aryl , arylene and alkylene groups of the composition of the present invention have from 2 to about 18 carbon atoms and most preferably 2 to about 12 carbon atoms.

A typical rubber composition in accordance with the present invention comprises a rubber, 0.1 to 5 phr of sulfur, 0.6 to 2 phr of a vulcanization accelerator, preferably a sulfenamide accelerator, 0.1 to 5 phr of the antidegradant and a C₁₂-C₂₀ fatty acid such as stearic acid. Metal oxides such as zinc oxide typically are added to rubber compositions.

The rubber composition of the present invention typically also comprises a reinforcing filler in a conventional amount. Any carbon black or combination of carbon black with any silica may be used.

Conventional rubber additives that may be included in the sulfur-vulcanizable rubber composition in accordance with the present invention include reinforcing agents such as carbon black, silica, clay, whiting and other mineral fillers, processing oils, tackifiers, waxes, phenolic antioxidants, phenylenediamine other antidegradants, antiozonants, pigments, e.g. titanium dioxide, resins, plasticizers, factices, and vulcanization activators, such as stearic acid and zinc oxide. These conventional rubber additives may be added in amounts known to the person skilled in the art of rubber compounding.

For further details on these typical rubber additives and vulcanization inhibitors, see W. Hofmann, Rubber Technology Handbook, Hanser Publishers, Munich 1989.

Finally, in specific applications it may also be desirable to include steel-cord adhesion promoters such as cobalt salts and bis-thiosulfates in conventional, known quantities.

A typical method of preparing a rubber composition comprises preparing a masterbatch consisting of rubber, carbon black, a vulcanization activator, antidegradant, and a processing oil, in an internal mixer such as a Banbury mixer or a Werner & Pfleiderer mixer. Subsequently, a vulcanization system is added to the masterbatch comprising sulfur and a vulcanization accelerator either in a low temperature mixer or on a two-roll mill, i.e. the productive stage of mixing. The uncured rubber composition is then vulcanized by heating, e.g., in a compression mold.

The composition of the present invention is useful in the manufacture of many articles, including pneumatic tires, e.g., for passenger cars and trucks, and industrial rubber goods, which comprise the rubber vulcanizate.

The compositions of the present invention may also function as inhibitors and serve to stabilize polymerization monomers during manufacturing steps and storage by minimizing polymer formation; these inhibitors must be removed prior to using the monomer for subsequent polymerization. Similarly, gasolines and lubricating oils may be stabilized to prevent formation of undesirable polymers that detract from product performance. When used in a controlled fashion, the composition of the present invention has a great practical significance stabilizing materials of commerce. Thereby, radical processes in elastomers, olefinic monomers, lubricating oils, and gasoline can be inhibited, retarded, and stopped.

The invention is illustrated by the following examples.

### EXAMPLES

Synthesis Method I

This method illustrates the general procedure to prepare 2-thio-substituted-1,4-benzoquinone intermediates where a stoichiometric amount of sodium periodate is employed. The appropriate 2-alkylthio-, 2-arylthio- or 2-heteroylthio-hydroquinone (10 mmole) was dissolved in 50 ml. methylene chloride and a solution of sodium periodate (10 mmole) in 10 ml. deionized water was added. Benzyltrimethylammonium bromide (50 mg.) was added and the mixture stirred at room temperature for 5 minutes. The organic layer was separated and evaporated under reduced pressure. Purification of the residue resulted in crystalline product. By this procedure, Methyl 3-[(3,6-dioxo-1,4-cyclohexadien-1-yl)sulfanyl]propanoate (A) and 2-(Cyclohexylsulfanyl)-1,4-benzoquinone (B) were prepared in 87% and 94% yields, respectively. Elemental analysis of A found C, 53.18% and H, 4.44% (theory: C, 53.09; and H, 4.45). Product (A) was isolated as orange needles (m.p. 91-94 C.) and (B) as orange plates m.p. (111-112 C.). The ¹H NMR and ¹³C NMR spectroscopic analyses of these intermediates were consistent with their chemical structures.

Synthesis Method 2

This method shows how to prepare the 2-thio-substituted-1,4-benzoquinone intermediates with catalytic (less than stoichiometric) quantities of sodium periodate. Reaction of 2-cyclohexylthio-hydroquinone (10 mmole) dissolved in 50 ml methylene chloride with hydrogen peroxide (10 mmole) in the presence of catalytic amounts of sodium periodate (1 mmole) and benzyltrimethylammonium bromide (50 mg.) gave the desired product, B, in 40% yield after just 10 minutes of stirring at room temperature. In contrast, no reaction took place with hydrogen peroxide in the absence of sodium periodate.

Synthesis Method 3

This method illustrates the general syntheses of 2-thio-5-amino-substituted benzoquinones where amine is added to the benzoquinone intermediate. The appropriate 2-alkylthio-, 2-arylthio- or 2-heteroylthio-1,4-benzoquinone (2 mmole) was dissolved in 25 ml. ethanol and treated with the corresponding amine (2 mmole) under a constant stream of air. The reaction medium turned dark purple on stirring at 20-25 C. for as little as 2 hours and as long as 20 hours. The solvent was evaporated and the resulting residue recrystallized from hexane and absolute alcohol to give the crystalline products described in Table 3.

Synthesis Method 4

Novel compositions of the present invention were prepared using the known process employing silver oxide. Thio-substituted-N-(1,3-dimethylbutyl)-N′-phenyl-p-quinonediimines were synthesized by oxidizing a toluene solution of the parent p-phenylenediamine with silver oxide in the presence of magnesium sulfate for as much as 20 hours over a 20-25 C. range. The resulting orange slurry was filtered, the filtrate evaporated, and the residue purified by chromatography to give a mixture of stereoisomers described in Table 6.

### Examples 1-12

Each of these Examples involved the preparation of 2-thio-5-amino-substituted benzoquinones from the corresponding 2-thio-substituted benzoquinone intermediates and the appropriate amine in the presence of air according to Synthesis Method 3. These novel unsymmetrical benzoquinones are summarized in Table 1 with their respective capacities as antioxidants as determined by oxidation induction times.

TABLE 1
Examples of 2-Thio-5-Amino-substituted Benzoquinones and
Their Respective Oxidation Induction Times (OIT)^{a}

| Example | | | OIT |
|---|---|---|---|
| No. | Thio Group | Amino Group | Minutes |
| 1 | Methyl 3-thiopropionate | N-Anilino | 25.0 |
| 2 | Methyl 3-thiopropionate | N-3-Anisidino | 33.6 |
| 3 | Methyl 3-thiopropionate | N-4-Isopropylanilino | 32.6 |
| 4 | Methyl 3-thiopropionate | N-2-Toluidino | 25.0 |
| 5 | Methyl 3-thiopropionate | N-2,4-Dimethylanilino | 14.6 |
| 6 | Methyl 3-thiopropionate | N-2-naphthylamino | 34.8 |
| 7 | Methyl 3-thiopropionate | N-4-Anilinoanilino | 243.1 |
| 8 | Methyl 3-thiopropionate | N-Phenethylamino | 12.2 |
| 9 | Cyclohexylthio | N-Anilino | 12.9 |
| 10 | Methyl 3-thiopropionate | N-Methyl-N-anilino | 13.7 |
| 11 | Methyl 3-thiopropionate | N-Morpholino | 10.3 |
| 12 | Methyl 3-thiopropionate | N-Indolinyl | 18.9 |

^{a}Oxidation induction times (OIT) were determined in polyisoprene at 160° C. and compared to both N-(1,3-dimethylbutyl)-N′-phenyl-p-phenylene diamine and N-(1,3-dimethylbutyl)-N′-phenyl-p-quinonediimine which under the same conditions exhibited OIT of 12.5 and 8.0 minutes, respectively.

The corresponding structures of the products of the above examples, 1-12, are shown in Table 2.

TABLE 2
2-Thio-5-amino-substituted Benzoquinones

| Example # | Compound |
|---|---|
| 1 | 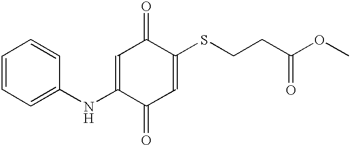 |
| 2 | 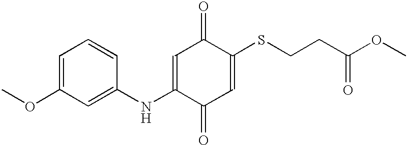 |
| 3 | 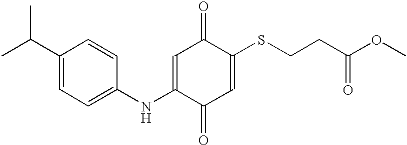 |
| 4 | 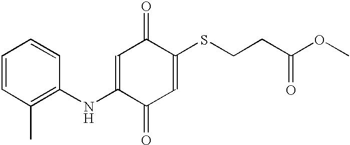 |
| 5 | 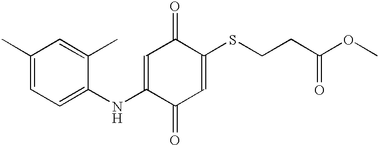 |
| 6 | 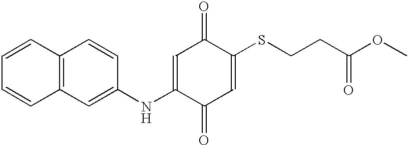 |
| 7 | 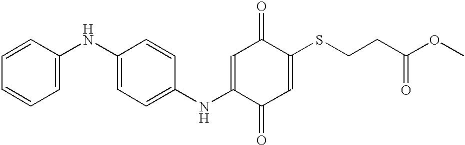 |
| 8 | 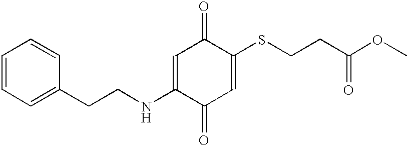 |
| 9 | 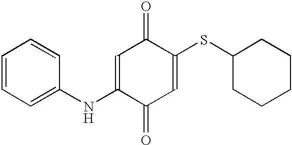 |
| 10 | 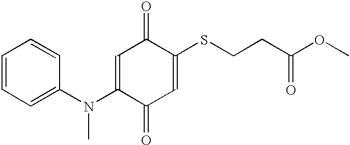 |
| 11 | 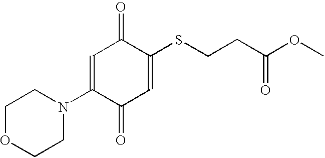 |
| 12 | 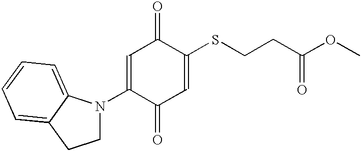 |

Table 3 itemizes the yields and analyses of the 2-Thio-5-amino-substituted Benzoquinones of Examples 1-12.

TABLE 3

| | | | State | N, Found |
|---|---|---|---|---|
| No. | Amine Derivative | Yield % | (m.p. ° C.) | (Theory)^{a} |
| 1 | Aniline | 90 | Purple solid | 4.29, (4.41) |
| | | | (180-181) | |
| 2 | m-Anisidine | 84 | Brown solid | 3.92, (4.03) |
| | | | (117-120) | |
| 3 | p-Isopropylaniline | 71 | Purple solid | 3.90, (3.90) |
| | | | (135-137) | |
| 4 | α-Toluidine | 75 | Red needles | 4.20, (4.23) |
| | | | (135-138) | |
| 5 | 2,4-Dimethylaniline | 92 | Red plates | 4.12, (4.05) |
| | | | (135-137) | |
| 6 | β-Naphthylamine | 85 | Black solid | 3.79, (3.81) |
| | | | (166-169) | |
| 7 | 4-Aminodiphenylamine | 97 | Purple solid | 6.96, (6.86) |
| | | | (206-208) | |
| 8 | Phenethylamine | 70 | Purple solid | N.A.^{b} |
| | | | (137-138) | |
| 9 | Aniline^{c} | 77 | Brown solid | 4.10, (4.47) |
| | | | (170-172) | |
| 10 | N-Methylaniline | 73 | Dark red solid | 4.16, (4.23) |
| | | | (113-115) | |
| 11 | Morpholine | 81 | Purple solid | 4.12, (4.50) |
| | | | (137-138) | |
| 12 | Indoline | 76 | Black oil | 3.87, (4.08) |

^{a}Spectroscopic analyses of Examples 1-12 by ¹H NMR and ¹³C NMR are consistent with the compound structures given in Table 2.
^{b}Elemental analysis for N not available, only C—62.93, (62.59); H—5.88, (5.54).
^{c}All examples contain a 2-thio group derived from methyl 3-mercaptopropionate except this example which contains a cyclohexylthio-group.

### Examples 13-21

Each of these examples was synthesized by oxidizing a toluene solution of the parent p-phenylenediamine with silver oxide in the presence of magnesium sulfate for as much as 20 hours over a 20-25 C. range. The resulting orange slurry was filtered, the filtrate evaporated, and the residue purified by chromatography to give a mixture of stereoisomers listed in Table 4 with their respective capacities as antioxidant as determined by oxidation induction times.

TABLE 4
Examples of Thio-substituted-N-(1,3-dimethylbutyl)-N′-phenyl-p-
quinonediimines and Their Respective Oxidation Induction Times (OIT)^{a}

| Example No. | Thio Group | OIT Minutes |
|---|---|---|
| 13 | Methyl 3-thiopropionate | 34.6 |
| 14 | Isopropylthio | 22.3 |
| 15 | Dodecylthio | 38.1 |
| 16 | 2-Pyrimidylthio | 7.7 |
| 17 | 2-Pyridinethio | 22.6 |
| 18 | n-Butylthio | 23.0 |
| 19 | Cyclohexylthio | 57.3 |
| 20 | t-Butylthio | 12.2 |
| 21 | 4,6-Dimethyl-2-pyrimidylthio | 3.5 |

^{a}Oxidation induction times (OIT) were determined in polyisoprene at 160° C. and compared to both N-(1,3-dimethylbutyl)-N′-phenyl-p-phenylene diamine and N-(1,3-dimethylbutyl)-N′-phenyl-p-quinonediimine which under the same conditions exhibited OIT of 12.5 and 8.0 minutes, respectively.

The corresponding structures of the products of the above examples, 13-21, are shown in Table 5.

TABLE 5
Thio-substituted-N-(1,3-dimethylbutyl)-N′-phenyl-
p-quinonediimines

| Example # | Compound |
|---|---|
| 13 | 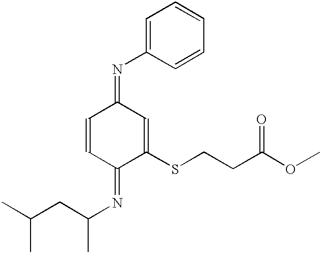 |
| 14 | 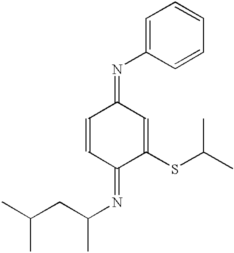 |
| 15 | 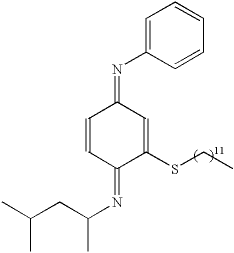 |
| 16 | 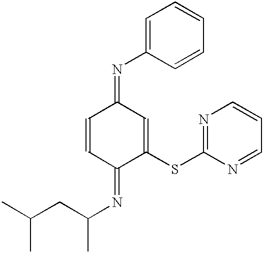 |
| 17 | 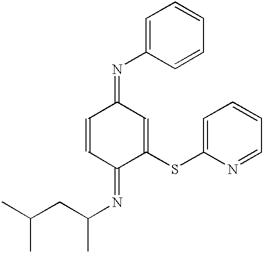 |
| 18 | 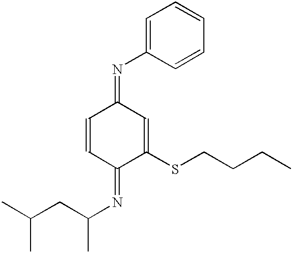 |
| 19 | 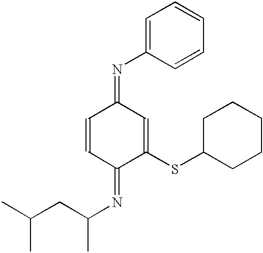 |
| 20 | 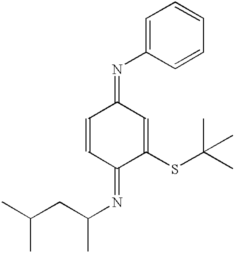 |
| 21 | 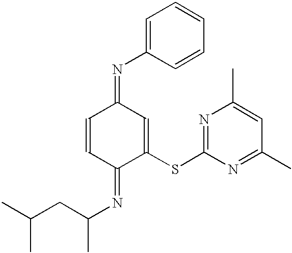 |

Table 6 itemizes the yields and analyses of the 2-Thio-substituted quinonediimines of Examples 13-21.

TABLE 6

| | | | State | N, Found |
|---|---|---|---|---|
| No. | Mercapto Derivative | Yield % | (m.p. ° C.) | (Theory)^{a} |
| 13 | Methyl | 99 | Orange oil | 6.79, (7.28) |
| | 3-mercaptopropionate | | | |
| 14 | Isopropyl mercaptan | 99 | Orange oil | 8.15, (8.23) |
| 15 | Dodecyl mercaptan | 88 | Orange oil | 6.27, (6.00) |
| 16 | 2-Mercaptopyrimidine | 80 | Orange oil | 14.51, (14.88) |
| 17 | 2-Mercaptopyridine | 55 | Orange oil | 11.22, (11.19) |
| 18 | n-Butyl mercaptan | 99 | Orange oil | 7.90, (8.26) |
| 19 | Cyclohexyl mercaptan | 88 | Orange oil | 6.94, (7.36) |
| 20 | t-Butyl mercaptan | 97 | Orange oil | 8.05, (7.90) |
| 21 | 4,6-Dimethyl-2- | 98 | Orange solid | 13.72, (13.85) |
| | mercaptopyrimidine | | 112-114 | |

^{a}Spectroscopic analyses of Examples 13-21 by ¹H NMR and ¹³C NMR and selected 10 elemental analyses (C, H, and N) are consistent with the compound structures given in Table 5.

Table 7 provides Examples of 2-Thio-5-Amino-substituted Benzoquinones and their Respective Oxidation Induction Times in Polybutadiene^{a}

TABLE 7

| Example | | | OIT |
|---|---|---|---|
| No. | Thio Group | Amino Group | Minutes |
| 1 | Methyl 3-thiopropionate | N-Anilino | 9.2 |
| 2 | Methyl 3-thiopropionate | N-3-Anisidino | 9.4 |
| 3 | Methyl 3-thiopropionate | N-4-Isopropylanilino | 8.4 |
| 4 | Methyl 3-thiopropionate | N-2-Toluidino | 11.0 |
| 5 | Methyl 3-thiopropionate | N-2,4-Dimethylanilino | 7.8 |
| 6 | Methyl 3-thiopropionate | N-2-naphthylamino | 12.8 |
| 7 | Methyl 3-thiopropionate | N-4-Anilinoanilino | 19.6 |
| 8 | Methyl 3-thiopropionate | N-Phenethylamino | 6.8 |
| 9 | Cyclohexylthio | N-Anilino | 5.6 |
| 10 | Methyl 3-thiopropionate | N-Methyl-N-anilino | 6.5 |
| 11 | Methyl 3-thiopropionate | N-Morpholino | 5.7 |
| 12 | Methyl 3-thiopropionate | N-Indolinyl | 7.1 |
| (A) | Methyl 3-thiopropionate | None | 2.9 |
| (B) | Cyclohexylthio | None | 4.9 |

^{a}Oxidation induction times (OIT) were determined in polybutadiene at 140° C. and compared to their precursors, Methyl 3-[(3,6-dioxo-1,4-cyclohexadien-1-yl)sulfanyl]propanoate (A) and 2-(Cyclohexylsulfanyl)-1,4-benzoquinone (B)

Table 8 shows oxidation Induction times of Thio-substituted-N-(1,3-dimethylbutyl)-N′-phenyl-p-quinonediimines in Polybutadiene^{a}

TABLE 8

| Example No. | Thio Group | OIT Minutes |
|---|---|---|
| 13 | Methyl 3-thiopropionate | 12.0 |
| 14 | Isopropylthio | 15.3 |
| 15 | Dodecylthio | 17.9 |
| 16 | 2-Pyrimidylthio | 12.1 |
| 17 | 2-Pyridinethio | 7.0 |
| 18 | n-Butylthio | 11.8 |
| 19 | Cyclohexylthio | 14.7 |
| 20 | t-Butylthio | 10.9 |
| 21 | 4,6-Dimethyl-2-pyrimidylthio | 3.7 |

^{a}Oxidation induction times (OIT) were determined in polybutadiene at 140° C. and compared to their precursor, N-(1,3-dimethylbutyl)-N′-phenyl-p-quinonediimine, which under the same conditions exhibited an OIT of 10.9 minutes.

The above data illustrate that in the overwhelming majority of cases where various embodiments of the compounds of the present invention are tested in polybutadiene or polyisoprene, the oxygen induction times obtained are superior (longer) as compared to that obtained when using known compounds

1. A process for preparing a compound having the formula comprising: 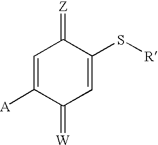
where
Z and W are the same or different, and selected from the group consisting of O, NH and NR, where R is H, alkyl, cycloalkyl or aryl, R′ is alkyl, cycloalkyl, alkylene, alkyl 3-propionate, aryl or arylene, and
where A is H if either or both of Z and W are not O, but if both Z and W are O, A is: 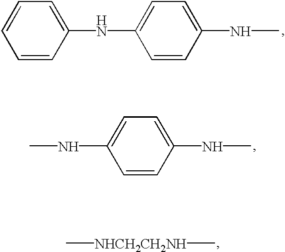
or a carbon based heterocyclic component having an amine constituent, or RR″N-, where R″ is the same or different than R and is selected from the same group as R, comprising preparing an alkylthio-substituted benzoquinone intermediate by reacting in a first reaction mixture a thio-substituted aromatic having the formula: 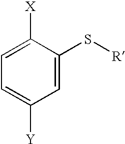
where X is OH, NH₂ or NHR when Z is O, NH or NR, respectively, and where Y is OH, NH₂ or NHR when W is O, NH or NR, respectively, with an alkali metal periodate in the presence of a phase transfer catalyst, followed, if A is not H, by reacting said intermediate in a second reaction mixture with an amine included in the group of amines comprising A, and in the presence of oxygen to obtain said compound, but if A is H no second reaction mixture is employed and said intermediate comprises said compound.

2. The process of claim 1 wherein said compound is a thio substituted amino-1,4-benzoquinone having the formula comprising 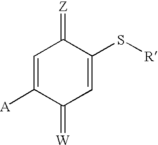
where A is: 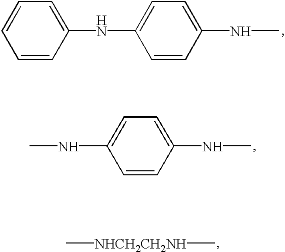
or a carbon based heterocyclic component having an amine constituent, or RR″N-, where R″ is the same or different than R and is selected from the same group as R, said thio-substituted amino-1,4-benzoquinone obtained by reacting a thio-substituted 1,4-benzoquinone with a primary or secondary amine to obtain said thio-substituted amino-1,4-benzoquinone in accordance with the equation:

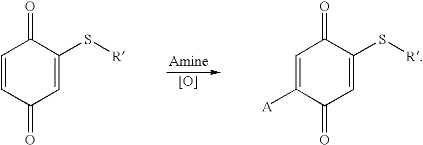

3. The process of claim 1 wherein the amine reacted is 80% or less of the 1:1 stoichiometric amount required to produce said thio-substituted amino-1,4-benzoquinones.

4. The process of claim 1 wherein the first reaction mixture includes a solvent in which the organic phase of the first reaction mixture is soluble and the first reaction mixture comprises an organic phase and an aqueous phase, and following the reaction of the first reaction mixture, the organic phase dissolved in said solvent is separated from the aqueous phase and comprises the second reaction mixture.

5. The process of claim 4 wherein said solvent is selected from the group consisting of methylene chloride, chlorobenzene, and C₁-C₄ alcohols.

6. The process of claim 5 wherein said solvent is selected from the group consisting of methanol, ethanol, and i-propyl alcohol.

7. The process of claim 1 wherein said alkali metal periodate may be used in either catalytic or stoichiometric amounts with respect to the thio-substituted aromatic.

8. The process of claim 7 wherein said alkali metal periodate is used in catalytic amounts and additional oxidant is added to the first reaction mixture in an amount sufficient to complete the oxidation of the thio-substituted aromatic.

9. The process of claim 8 wherein said additional oxidant is hydrogen peroxide.

10. The process of claim 1 wherein the alkali metal periodate is sodium periodate.

11. The process of claim 1 wherein said first reaction mixture includes methylene chloride solvent, the phase transfer catalyst comprises benzyltrimethylammonium bromide, the alkali metal periodate is sodium periodate which is used in either stoichiometric or catalytic amounts with respect to the thio-substituted aromatic.

12. The process of claim 11 wherein the sodium periodate is used in catalytic amounts with hydrogen peroxide as the additional oxidant in an amount sufficient to complete the oxidation of said thio-substituted aromatic.

13. The process of claim 1 wherein the reaction conditions used with the first and second reaction mixtures comprise a temperature of from about 20° C. to about 25° C., the residence time for the first reaction mixture is from about 5 minutes to about 2 hours, the residence time for the second reaction mixture is from about 2 hours to about 4 hours and the oxygen for the second reaction mixture is provided by a constant stream of air.

14. The process of claim 1 wherein the amine contained in said second reaction mixture is selected from the group consisting of aniline, 3-anisidine, 4-isopropylaniline, 2-toluidine, 2,4-dimethylaniline, 2-naphthylamine, 4-anilinoaniline, phenethylamine, N-methyl-aniline, morpholine, and indoline.

15. A composition comprising a compound having the formula comprising: 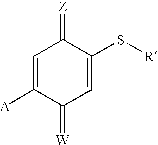
where Z and W are the same or different, and selected from O, NH, NR, where R is H, alkyl, cycloalkyl or aryl, R′ is alkyl, cycloalkyl, alkylene, alkyl 3-propionate, aryl or arylene, and where A is H if either or both of Z and W are not O but if both Z and W are O, A is: 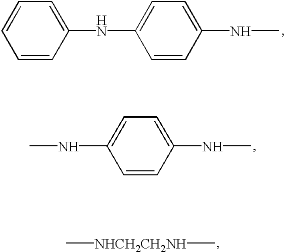
or a carbon based heterocyclic component having an amine constituent, or RR″N-, where R″ is the same or different than R and is selected from the same group as R.

16. Compounds of the composition of claim 15 comprising a thio-substituted amino-1,4-benzoquinone having the formula:

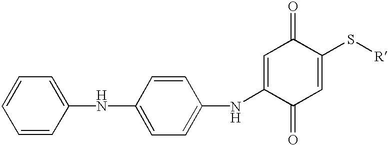

17. The composition of claim 15 wherein said compound is selected from the group consisting of thio-amino-substituted 1,4-benzoquinones, thio-substituted 1,4-benzoquinoneimines, and thio-substituted 1,4-benzoquinonediimines.

18. The composition of claim 15 wherein said compound is an amino-1,4-benzoquinone having the formula comprising: 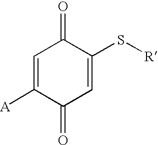
where A is: 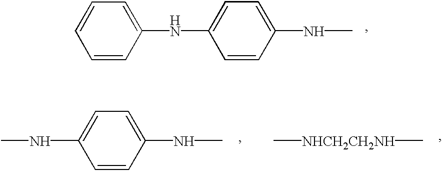
or a carbon based heterocyclic component having an amine constituent, or RR″N, where R″ is the same or different than R and is selected from the same group as R.

19. A composition comprising natural or synthetic rubber or blend thereof and an antidegradant comprising one or more compounds of claim 15.

20. The composition of claim 19 comprising polybutadiene or polyisoprene rubber.

21. A composition comprising natural or synthetic rubber or a blend thereof and an antidegradant comprising a compound of claim 15 and one or more non-thio-substituted antidegradants selected from the group consisting of phenylenediamines, dihydroquinolines, and phenolics, or a blend thereof.

22. A composition comprising a monomer for subsequent polymerization, gasoline or lubricating oil containing a compound of claim 15 as a polymerization inhibitor.

23. A composition comprising natural or synthetic rubber or a blend thereof and an antidegradant selected from the group consisting of thio-substituted amino-1,4-benzoquinones, thio-substituted 1,4-benzoquinoneimines, and thio-substituted 1,4-benzoquinonediimines.

24. A composition comprising natural or synthetic rubber or a blend thereof and an antidegradant selected from the group consisting of 2-(4-anilinoanilino)-5-(cyclohexylsulfanyl)benzo-1,4-quinone, and 3-{[4-(4-anilinoanilino)-3,6-dioxo-1,4-cyclohexadien-1-yl]sulfanyl}propanoate, or a mixture thereof.
